(19) United States
(12) Patent Application Publication
(10) Pub. No.: US 2003/0166274 A1
(43) Pub. Date: Sep. 4, 2003

(54) Three-dimensional matrix for producing living tissue equivalents

(76) Inventors: Charles W. Hewitt, Blackwood, NJ (US); Louise Strande, Voorhees, NJ (US); Kevin M. Schuster, Collingswood, NJ (US)

Correspondence Address: PERKINS COIE LLP, POST OFFICE BOX 1208, SEATTLE, WA, 98111-1208, US

(21) Appl. No.: 10/295,398

(22) Filed: Nov. 15, 2002

Related U.S. Application Data
(60) Provisional application No. 60/332,138, filed on Nov. 15, 2001.

(51) Int. Cl. C12N 5/08

(52) U.S. Cl. 435/366

(57) ABSTRACT

The primary aspect of the present invention it directed to a three-dimensional matrix, a living tissue equivalent, a tissue microarray, methods of making thereof, and methods of using thereof. In one embodiment of the invention, the three-dimensional matrix comprises fibroblasts and a fibrin matrix. In a preferred embodiment, the three-dimensional matrix comprises blood plasma, thrombin and fibroblasts. The three-dimensional matrix in accordance with this invention may be used to construct living tissue equivalents, including but not limited to skin, blood vessel, bone, tendon, ligaments, and organ equivalents, among many others. Furthermore, the living tissue equivalents may be used to test the effect of various agents and to study the mechanism of disease and the efficacy of various treatment protocols.

### RELATED APPLICATION

This application claims the benefit of U.S. Provisional Application No. 60/332,138, which was filed on Nov. 15, 2001.

### FIELD OF THE INVENTION

The present invention relates to a method of making an in vitro three-dimensional matrix for use in constructing living tissue equivalents, generating tissue microarrays, growing genetically engineered cells and/or tissue equivalents and assaying the interaction between a living tissue equivalent and various agents.

### BACKGROUND OF THE INVENTION

Every year, millions of people in the United States suffer from tissue or organ injury or loss due to disease, accident, congenital abnormalities, and defects. Tissue injury, such as burns, venous leg ulcers, diabetic foot ulcers, vascular injuries, bone loss and cartilage injury require immediate medical intervention. But the success of medical treatment is often limited by the availability of donor organs, the danger of rejection, disease transmission, and the inability of mechanical or artificial tissue or organ to perform all of the natural biological functions.

Surgical skin grafts are often required to treat venous leg ulcers, diabetic foot ulcers and skin burns. If left untreated, these injuries may lead to infections and more serious complications. For example, diabetic foot ulcers may require the amputation of a toe, a foot, or an entire limb. Accordingly, there is an immediate need for skin to cover the wounded area.

There is also a need for vascular grafts. Approximately 350,000 coronary bypass grafting procedures are performed annually in the United States alone. Typically, coronary bypass grafting is performed by harvesting a vein from the patient's leg and implanting it in place of the blocked artery. But only a limited amount of venous material may be harvested from a single patient. Hence, a patient needing multiple arterial grafts may not have enough venous material. Therefore, blood vessel equivalents could replace the need to use patient vein for grafting material.

The best tissue equivalent is one that is closest to native human tissue in histological and functional properties. An autologous tissue replacement, comprised of the patient's own cells, would be the replacement of choice. But in many cases, the deficiency of autologous tissue has yet to be overcome to make transplantation or implantation of autologous tissue a feasible method of treatment. Allogenic tissue replacement may solve the problem of tissue deficiency, but it presents the risk of rejection and disease transmission, such as cytomegalovirus, human immunodeficiency virus (HIV), hepatitis B virus, and hepatitis C virus, to name a few. Accordingly, the concept of restoring tissue with bio-materials that mimic natural tissue has been studied extensively.

Advances in tissue engineering bring the promise of developing effective tissue equivalents that can restore, maintain or improve the function of damaged tissues and organs, or even replace lost or damaged tissues and organs. But while major developments have been made in the field of tissue engineering, there is constant need for improving both the tissue equivalents and the processes for making and using them.

The science of tissue-engineering involves the interactions of three components: cells, a matrix, and growth factors. Collagen is one of the main structural fibers for tissues and organs. Also, collagen is the most abundant of all fibers for tissues, such as skin, bone, tendon, ligaments, dentine, sclera, fascia, and organ capsules. Additionally, collagen has been used in a wide variety of applications in the manufacture of prosthesis as well as in the preparation of living tissue equivalents. Much work has been done to develop collagen constructs for such applications, including constructs for use in research and development, tissue and organ repair or replacement.

One of the key issues in tissue engineering is selecting the matrix material. Tissue engineers have focused on the use of polymers and exogenous collagen. However, when materials are not derived directly from the patient, extensive testing and treatment is necessary to ensure that antigens and pathogens are not present. The challenges of developing a suitable matrix are exemplified by past efforts at developing a composite human skin equivalent.

The skin is the largest organ of the human body. It consists of two layers: the superficial epidermis, which itself is made up of several layers of keratinocytes, and a deep layer, the dermis, which is composed of a structural collagen matrix.

One of the first major advances in the field of living skin equivalents was the production of large surfaces of the epidermal layer from relatively small areas of the patient's own skin through the in vitro cultivation of epidermal cells. An early development of this technique was carried out by Rheinwald and Green in 1975, who co-cultivated irradiated fibroblasts with human keratinocytes. Rheinwald J G, et al. Serial cultivation of strains of human epidermal keratinocytes: the formation of keratinizing colonies from single cells. Cell. 1975; 6:331-334. Improvements on this technique have led to the production of a graftable epidermal sheet that may be 1,000- to 10,000-fold larger than the initial tissue biopsy obtained from the donor patient. Rouabhia M. Skin Substitute Production by Tissue Engineering: Clinical and Fundamental Applications. Austin, Tex.: R. G. Landes Company 1997. However, the presence of foreign proteins in the epithelial cell culture in preparing living skin equivalents may cause an immune reaction. Thus, further developments were directed at eliminating, or at least reducing, the foreign proteins in the epithelial cell culture.

Notwithstanding the success with constructing an epidermis with cultured keratinocytes, the problem of constructing an autologous dermal matrix remained unresolved. The absence of a dermis was considered the major cause of epidermal graft rejection, as well as higher fragility and blistering after transplantation. Rouabhia M. Skin Substitute Production by Tissue Engineering: Clinical and Fundamental Applications. Austin, Tex.: R. G. Landes Company 1997. A dermis could promote graft take of cultured epidermal cells and prevent rejection of the epithelial cells. Hence, considerable efforts have been aimed at developing a non-immunogenic artificial dermal matrix. Approaches to constructing artificial dermal matrices have included constructing a collagen gel from bovine collagen, as disclosed in Bell E, Erhlich H P, Buttle D J et al. A living tissue formed in vitro and accepted as a full thickness tissue equivalent. Science 1981; 211:1042-1054 and using a synthetic mesh composed of a Nylon or a polyglactic acid mesh on which fibroblasts are cultured.

The ability to produce true epidermal-dermal composite grafts in vitro would be a monumental leap forward in the treatment of burns, venous leg and diabetic foot ulcers. Many of the current in vitro skin constructs generally involve the use of collagen matrices, wherein the collagen is derived from exogenous sources, such as bovine, rat-tail, and cadaverous tissues. Hence, the exogenous collagen that is used for tissue engineering must be modified to reduce its immunogenicity through sterilization methods that preserve its biological and physical properties. The need for such a sterilant has resulted in the development of a variety of different methods. However, many of the sterilization techniques are only partially effective and may alter the essential physical and biological characteristics of collagen. Nevertheless, even modified collagen presents a risk of eliciting an immunogenic response in the graft recipient.

It would be ideal to develop a living tissue equivalent that did not rely on a matrix containing exogenous collagen. An ideal matrix would be produced de novo using human cells only. Development of an autologous three-dimensional collagen matrix however, has yet to be achieved using current techniques.

In addition to the need for human tissue equivalents, there is a scarcity of eukaryotic expression systems comprised of human allogenic or autologous cells or tissue that are capable of large-scale protein production. Currently, eukaryotic cells grown in culture are maintained as monolayers in tissue culture flasks or dishes in a two-dimensional format. Such a format results both in low cell number:media volume ratios and protein yields. Moreover, cells grown in such a fashion are often “transformed” such that they lack the normal controls on cellular growth and constantly enter the “S” phase of cell division. They are unsuitable for human implantation as they may become cancerous once inside the body. In addition, the cellular structure, function or protein expression profile may change as a result of the transformation or immortalization, rendering the cells unsuitable for their intended purpose.

### SUMMARY OF THE INVENTION

The present invention satisfies a long-felt need for a three-dimensional matrix that is produced de novo using human cells only. Human fibroblasts are capable of producing collagen and components of the extracellular matrix in vitro. Accordingly, the present invention provides a method of producing a three-dimensional collagen matrix from human fibroblasts for the production of living tissue equivalents, such as skin, blood vessel, bone, tendon, ligaments, and organ equivalents, among many others. In a preferred embodiment, the human fibroblasts are autologous.

An advantage of the present invention is that it harnesses the body's own response to injury, a hemostatic clot, as a substrate to form a three-dimensional collagen matrix. A hemostatic clot is formed by a mixture of blood plasma and thrombin. Blood plasma contains fibrinogen, which polymerizes and cross-links to produce an insoluble fibrin matrix in the presence of thrombin. Accordingly, in a preferred embodiment, a three-dimensional collagen matrix is formed by including fibroblasts in a hemostatic clot mixture comprising blood plasma and thrombin, wherein the fibrinogen polymerizes and cross-links to produce an insoluble fibrin matrix (hemostatic clot) and the fibroblasts proliferate and produce collagen. Optionally, platelets, clotting factors, growth factors, fibronectin, and/or von-Willebrand factor are added to the hemostatic clot mixture. In another embodiment, other cells may be included in the hemostatic clot mixture. Preferably, nutrients or growth media are provided to maintain the fibroblasts and/or other cells within the hemostatic clot mixture.

It is known that fibroblasts produce different collagen types depending on the tissue from which they are derived. For example, collagen type I accounts for 90% of body collagen and is found in tissues, such as skin, bone, tendon, ligaments, dentine, sclera, fascia, and organ capsules. Collagen type II is found in cartilage, notochord, and intervertebral disc. Collagen type III is found in the connective tissue of organs, such as the uterus, liver, spleen, kidney, and lungs. In short, as many as 16 different types of collagen have been identified.

Fibroblasts are also known to produce different fiber types, such as reticular and elastic fibers. Thus, in one preferred embodiment of the present invention, the proportions and types of collagen or other fibers desired for the three-dimensional matrix may be manipulated by selecting the appropriate fibroblasts that will produce the desired collagen types or other fibers.

The desired characteristics of the resulting three-dimensional matrix may also be selected by the adding growth factors in various combinations and concentrations. Growth factors may be added to increase fibroblast replication and collagen deposition. These growth factors include, but are not limited to, transforming growth factor beta (TGF-β), platelet derived growth factor (PDGF), epidermal growth factor (EGF), vascular endothelial growth factor (VEGF), cytokines IL-1 and TNF, and vitamin C.

In one embodiment, a three-dimensional collagen matrix may be used to culture various cells, such as keratinocytes, hepatocytes and bone marrow cells, to name a few.

In another embodiment, the three-dimensional matrix of the present invention may further be used to produce a living tissue equivalent. For example, a living tissue equivalent may be produced by including fibroblasts and/or other tissue or organ cells in the hemostatic clot mixture. Furthermore, the living tissue equivalents may be formed in any desired shape by forming the three-dimensional matrix within a mold or by impregnating a biocompatible scaffolding material. Examples of such living tissue equivalents may include, but are not limited to, blood vessels, bone, cartilage, tendon, ligament, and organ tissue equivalents.

Living tissue equivalents formed in accordance with the present invention may also be used in a wide variety of applications. The living tissue equivalents may be implanted directly into a patient to replace lost or damaged tissue. Also, the living tissue equivalents of the present invention may be used in connection with a tissue microarray as a model to study the effect of chemical, biological, synthetic, or other agents on a various tissues and organs. Living tissue equivalents may also be used to study the mechanism of diseases and the efficacy of drugs to treat them.

In one embodiment of the present invention, a tissue equivalent derived from the three-dimensional matrix may be used in a tissue microarray. Tissue microarrays provide a new method for high-throughput molecular profiling of tissue specimens or cells. (Kononen et al., Nature Medicine, 4:844-847 [1998]). Tissue microarrays provide a means for rapid, very large-scale molecular analysis. Tissue sections may easily be prepared using methods known to those skilled in the art. These microarrays may then be used in applications that include, but are not limited to, those that for probe for DNA, RNA, protein, lipid, and other intracellular or extracellular molecular targets. In addition, the microarray sections may be utilized for microscopic morphological analysis.

In another embodiment of the present invention, cells or tissues grown in the three-dimensional matrix may be genetically engineered. For example, a sample of a patient's liver cells could be genetically engineered to express increased levels of LDL receptor. These cells could be grown as a living tissue equivalent in the three-dimensional matrix. If the living tissue equivalent were implanted back into the patient, the genetically engineered tissue bearing increased an number of LDL receptors may lead to decreased serum cholesterol levels. There are a number of methods known to those skilled in the art of introducing genetic material into cells and tissues.

In yet another embodiment, cells grown in the three-dimensional matrix could be used to harvest and isolate proteins, produced by a patient's own cells, in large quantities. Large-scale production of proteins bearing cell-specific processing and assembly is an area of tremendous need. Such proteins would not be rejected by the patient and would bear proper tissue and cell-specific folding and processing.

Current methods of eukaryotic protein expression not only lack this capability but also have substantial drawbacks. Bacterial transformation results in large protein yields, but prokaryotic expressed proteins often lack proper folding, glycosylation and phosphorylation processing, resulting in possible rejection and lack of function. To overcome some of these problems, baculovirus systems using eukaryotic insect Sf9 and Sf21 cells (Invitrogen, Carlsbad, Calif.) to produce proteins have been employed, but they result in relatively low protein yields and the proteins lack mammalian glycosylation and processing patterns. The Sindbis Expression System (Invitrogen, Carlsbad, Calif.) has been used to transfect mammalian cells, but it results in transient expression and may therefore be unsuitable for long-term large-scale protein production. Chinese Hamster Ovary (CHO) cells are most commonly used to produce large amounts of eukaryotic proteins. To accomplish this goal, CHO cells are often injected with DNA, which is then transcribed and translated producing a large amount of protein relative to other available eukaryotic expression systems. This system, however, lacks the cell-specific folding and processing that a patient's own cells could provide. Moreover, if a patient required long-term implantation of genetically engineered tissue that secreted a defined protein, the use of CHO cells would be inappropriate, as the host would quickly reject them. The use of transgenic plants to produce antibodies used to fight disease has been described in U.S. Pat. No. 6,417,429. While capable of large-scale protein production, it is not clear that this method dutifully replicates mammalian-specific, much less tissue and cell-specific, processing of many eukaryotic secreted proteins. As a result, such proteins may lack optimum, or even normal, function. In addition, the host may quickly reject the proteins as foreign.

Cells grown in the three-dimensional matrix could be derived from virtually any tissue, resulting in species, tissue, and cell-specific expression of proteins. Moreover, cells could be taken from a patient, grown in the three-dimensional matrix, transfected with a gene of interest, and the desired protein could be harvested and isolated for use in the same patient. Alternatively, those cells that contain the gene of interest and produce the desired protein could be implanted back into the patient with a minimal chance of rejection.

### DESCRIPTION OF THE DRAWINGS

FIG. 1 is a digital image depicting an H&E stained platelet gel matrix populated with fibroblasts at a magnification of 100×.

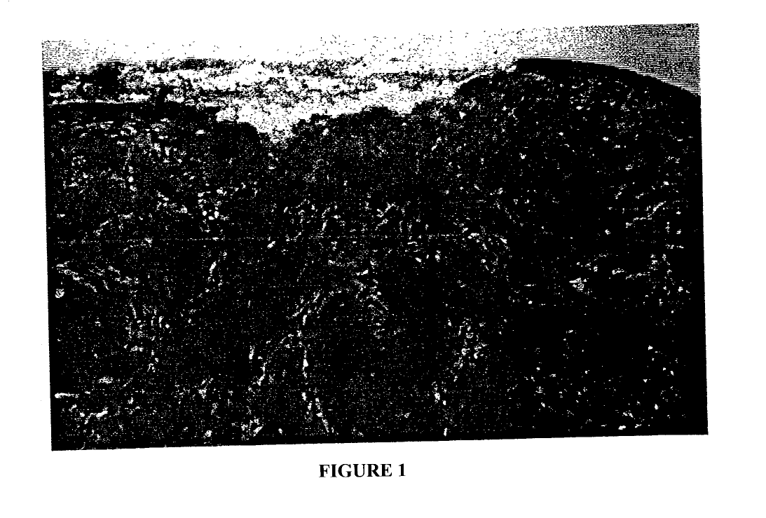

FIG. 2 is another digital image depicting an H&E stained the platelet gel matrix populated with fibroblast at a magnification of 400×.

FIG. 3 is a digital image showing the presence of collagen type I in the fibroblast populated platelet gel matrix.

FIG. 4 is a digital image showing the presence of collagen type III in the fibroblast populated platelet gel matrix.

FIG. 5 is a digital image displaying an H&E stained platelet matrix populated with fibroblasts and surface keratinocytes at a magnification of 100×.

FIG. 6 is a digital image displaying an H&E stained platelet matrix populated with fibroblasts and surface keratinocytes at a magnification of 400×.

### DETAILED DESCRIPTION OF THE INVENTION

The present invention provides a method of producing an in vitro three-dimensional collagen matrix for use in living tissue equivalents and also to assays for determining the interaction between a living tissue equivalent and various agents.

Preferably, the three-dimensional matrix is formed by including fibroblasts in a hemostatic clot mixture comprising blood plasma and thrombin, wherein the fibrinogen in the blood plasma polymerizes and cross-links to produce an insoluble fibrin matrix and the fibroblasts proliferate and produce collagen. In another embodiment, other cells may be included in the hemostatic clot mixture. Nutrients or growth media may be provided to maintain the fibroblasts and/or other cells within the hemostatic clot mixture.

The formation and characteristics of the hemostatic clot may be modulated in a number of ways. Preferably, the present invention includes the addition of platelets, clotting factors and/or growth factors to accelerate the formation of or to enhance the characteristics of the three-dimensional matrix. Platelets may be added to the mixture to accelerate the formation of or to produce a thicker three-dimensional matrix. Furthermore, the stability of the three-dimensional matrix may be enhanced by the addition of fibronectin, and/or von-Willebrand factor.

Fibroblasts are included in the hemostatic clot mixture to produce collagen in the three-dimensional matrix. The fibroblasts are resident cells of the connective tissue and they are responsible for the synthesis of collagen, elastic, and recticular fibers, as well as other components of the extracellular matrix, including proteoglycans and hyaluronic acid. Fibroblasts also have a central role in forming, maintaining, and turning over the extracellular matrix.

It is known that fibroblasts elaborate different collagen types and other fibers, such as reticular and elastic fibers, depending on the tissue from which they are derived. Thus, in one embodiment, the proportions and types of collagen or other fibers desired for the three-dimensional matrix may be manipulated by selecting the fibroblasts that will produce the desired collagen types or other fibers. For example, fibroblasts obtained from a patient's dermis may be included in the hemostatic clot mixture to produce collagen types I and III, both of which are necessary to produce a human skin equivalent. A three-dimensional matrix produced from a patient's own fibroblasts obviates the need to use collagen from an exogenous source. Thus, a skin equivalent comprising a matrix containing collagen produced by a patient's own cells will diminish the likelihood of tissue rejection and disease transmission.

Fibroblasts may be obtained from an organ or tissue using techniques known to those skilled in the art. For example, fibroblasts may be isolated by mechanically disaggregating an organ or tissue. Alternatively, fibroblasts may be isolated by enzymatically dissociating the tissue with digestive enzymes, such as trypsin, chymotrypsin, collagenase, elastase, and/or hyaluronase. The isolation of fibroblast by enzymatic dissociation may be performed by washing fresh, minced tissue samples in Hank's balanced salt solution (HBSS) to remove serum. The minced tissue may then be incubated in a solution of dissociating enzyme. After incubation, the dissociated cells may be suspended, pelleted by centrifugation and plated onto culture dishes. Furthermore, fibroblasts may be obtained from fibroblast stem cells. In this case, the fibroblast stem cells are mixed with a hemostatic clot mixture comprising blood plasma and thrombin and allowed to grow into fibroblasts in a growth media.

Collagen synthesis involves events that occur both within and outside of the fibroblast. The stimulation of fibroblasts is driven by the various growth factors to modulate collagen synthesis. In a preferred embodiment of the present invention, various growth and other regulatory factors are added to promote, inhibit, or modulate the growth of the fibroblasts to produce the collagen for the three-dimensional matrix. Accordingly, the characteristics of the three-dimensional matrix can be modified by the addition of growth factors in various combinations and concentrations. Such growth factors include, but are not limited to, transforming growth factor beta (TGF-β), platelet-derived growth factor (PDGF), fibroblast growth factor (FGF), epidermal growth factor (EGF), vascular endothelial growth factor (VEGF), cytokines IL-1 and TNF, and ascorbate (vitamin C).

For example, TGF-β and PDGF were found to stimulate the production of collagen by fibroblasts in the hemostatic clot. Also, TGF-β was found to inhibit rapid multiplication of fibroblasts, thereby preventing the destruction of the hemostatic clot. Vitamin C may optionally be added, since it is involved in the post-translational hydroxylation of proline and lysine, the hydrogen bonds required in forming the final structure of the collagen molecule.

In a preferred embodiment of the present invention, a living tissue equivalent may be produced from a three-dimensional matrix. A living tissue equivalent formed from autologous cells would significantly reduce the risk of rejection and disease transmission. In one embodiment, the present invention provides for a method for producing an in vitro living skin equivalent constructed from an autologous collagen.

In one embodiment, the three-dimensional matrix of the present invention may be used to produce living tissue equivalents, such as skin, blood vessel, bone, tendon, ligaments, and organ equivalents, among many others, by including fibroblast and other cells in the hemostatic clot mixture. Living tissue equivalents can be formed in any desired shape by forming the three-dimensional matrix in a mold or by impregnating a biocompatible scaffolding material. It is preferred a desired shape be the shape of a blood vessel, a bone, a bone marrow, a brain, a cartilage, a connective tissue, an eye, an ear, an endocrine organ, an epithelial tissue, a muscle tissue, a hair, a heart, an intestine, a lung, a liver, a kidney, a mouth, a skin, a nail, a nose, a nerve tissue, a pancreas, a respiratory duct, a stomach, an urinary bladder, a spinal cord, a tongue, a tooth, and a reproductive organ. The biocompatible scaffolding material may be formed into a mesh, woven, braided, knitted, or otherwise constructed to form the desired shape. The biocompatible scaffolding material may be biodegradable or non-biodegradable, depending on its intended use. For example, a biodegradable mesh may be used to blood vessel equivalents by impregnating it with the hemostatic clot mixture containing the other cells that exist in the artery, such as fibroblasts and smooth muscle cells. Once impregnated, the fibroblasts and smooth muscle may the collagen and elastin necessary to maintain the framework of the blood vessel equivalents. On the other hand, because cartilage and bone tissue are subjected to mechanical and load bearing stresses, cartilage and bone tissue equivalents may require the use of nonbiodegradable materials for added support.

The three-dimensional collagen matrix of the present invention may also be used to culture cells. For example, keratinocytes, the specialized cells of the skin, have been shown to proliferate and fully differentiate into a complete epidermal layer on the three-dimensional collagen matrix of the present invention. Similarly, other cells, such as hepatocytes and bone marrow cells, may be cultured on the three-dimensional collagen matrix of the present invention. In addition, cells to be culture in the matrix can be obtained from any tissue to organ. Furthermore, cells can be stem cells including epithelial tissue stem cells, connective tissue stem cells, muscle tissue stem cells and nervous tissue stem cells.

The living tissue equivalents herein disclosed also have a broad range of applications including applications in research and development, tissue and organ replacement, and also for the testing and screening of various chemical, biological, environmental, synthetic, or other agents. The effects of these agents on humans are of widespread concern. Accordingly, the present invention may provide for assays and kits for testing the effects of biological, chemical, and other agents on human tissue. Pharmaceutical, cosmetic, and chemical companies, research laboratories and governmental agencies may use these tests on living tissue equivalents to study the reaction of a particular tissue or organ equivalent to a particular agent(s). Therefore, the use of living tissue equivalents could obviate the need for human or animal subjects for the study of the effects of various harmful pharmacological, chemical, biological, synthetic, and other agents on the selected tissue.

Furthermore, the living tissue equivalents may be used to study the mechanism of disease and to further study the efficacy of various treatment protocols. In one embodiment of the present invention, living tissue equivalents may serve as a reliable model to evaluate tissue pathology and the mechanism of tissue or organ disease and injury. For example, the three-dimensional matrix may additionally include cells from a diseased tissue or organ to produce an equivalent of the diseased tissue for use in research. In another embodiment, both normal and diseased cells may be included in the three-dimensional matrix to serve as a model in determining the efficacy of drugs or other treatment protocols in targeting the diseased cells, while allowing the normal cells to remain intact.

In another embodiment of the present invention, a variety of tumor and other cells may be included in the three-dimensional matrix to study the mechanisms of tumor growth and metastasis and to further study the efficacy of chemotherapeutic agents on both tumor and normal cells. To this end, tumor cells derived from the biopsy sample of a patient may be included n the three-dimensional matrix to form a living tissue equivalent as a model of the diseased tissue. The tumor model may then be used to create an effective and individualized chemotherapy treatment for the patient that selectively targets malignant and diseased cells.

In another embodiment of the present invention, tissue microarrays may be created using tissue sections derived from the three-dimensional matrix. Tissue sections may be prepared using methods known to those skilled in the art, such as paraffin-embedding and frozen section preparation.

Small tissue core biopsies may be obtained from paraffin-embedded or frozen blocks and placed into recipient blocks in a format such that each sample occupies a defined coordinate, has a defined diameter and is a defined distance from all other coordinates. The blocks may then be sliced using conventional means (i.e. microtomes) and the tissue microarray may then be used in applications including, but not limited to, those that probe for DNA, RNA, protein, lipid, and other intracellular or extracellular molecular targets. In addition, the microarray sections may be utilized for microscopic morphological analysis.

In yet another embodiment of the present invention, cells or tissues grown in the three-dimensional matrix may be genetically engineered to express exogenously introduced genetic material. There are a number of methods known to those skilled in the art to introduce exogenous genetic material into a cell or tissue. These techniques may be designed to express a gene, temporarily or permanently, using transient or stable, transfection methods, respectively.

For example, a DNA element known to enhance the transcription of a cis-linked gene, such as a cytomegalovirus (CMV) element, may be linked to the coding region of a gene of interest. This DNA construct may be introduced into the cell using a variety of methods including, but not limited to, calcium phosphate transfection, cationic lipid transfection, viral insertion, DEAE-Dextran, microinjection and biolistic particle delivery. Once introduced into the cell, the DNA construct may increase the expression of the gene of interest resulting in a protein that may alter the structure or function of the cell. Moreover, it may alter the diagnostic and/or morphological characteristics of the cell or tissue.

Cells grown in the three-dimensional matrix could be used to harvest and isolate proteins, produced by a patient's own cells, in large quantities. Large-scale production of proteins bearing cell-specific processing and assembly is an area of tremendous need. Such proteins would bear a patient's own tissue and cell-specific processing patterns, resulting in normal protein function and a decreased risk of rejection. Current methods of eukaryotic protein expression not only lack this possibility, but have substantial drawbacks.

Bacterial transformation results in large protein yields, but prokaryotic expressed proteins often lack proper folding, glycosylation and phosphorylation processing, resulting in a lack of function and/or acceptance by the host. Baculovirus systems use Sf9 and Sf21 insect cells (Invitrogen, Carlsbad, Calif.) and have been employed to overcome some of these problems, but they result in relatively low protein yields and the proteins lack mammalian glycosylation and processing patterns, hindering proper function and possibly resulting in host rejection. The Sindbis Expression System (Invitrogen, Carlsbad, Calif.) has been used to transfect mammalian cells, but it results in transient expression and may therefore be unsuitable for long-term large-scale protein production, especially if the cells are intended for implantation. Chinese Hamster Ovary (CHO) cells are most commonly used to produce large amounts of eukaryotic proteins. To accomplish this goal, CHO cells are injected with DNA, which is transcribed and translated producing a large amount of protein relative to other eukaryotic expression systems. This system, however, lacks the cell-specific folding and processing that a patient's own cells could provide. Moreover, if a patient required long-term implantation of genetically engineered tissue that secreted a defined protein, the use of CHO cells would be inappropriate, as the host would quickly reject them. The use of transgenic plants to produce antibodies that could be used to fight disease has been described in U.S. Pat. No. 6,417,429. It is not clear that this method, while capable of large-scale protein production, can dutifully replicate mammalian-specific, much less the tissue and cell-specific processing of many eukaryotic secreted proteins. As a result, such proteins may lack optimum, or even normal, function. In addition, the host may quickly reject the proteins as foreign.

Cells grown in the three-dimensional matrix could be derived from virtually any tissue, resulting in species, tissue, and cell-specific expression of proteins. Moreover, cells could be taken from a patient, grown in the three-dimensional matrix, transfected with a gene of interest, and the desired protein could be harvested and isolated for use in the same patient. Alternatively, those cells that contain the gene of interest and produce the desired protein could be implanted back into the patient with a minimal chance of rejection.

These descriptions are provided only as examples and should not be understood to be limiting on the claims. Based on the description, a person of ordinary skill in the art may make modifications and changes to the preferred embodiments, which does not depart from the scope of the present invention.

### EXAMPLE I

### Method of Producing a Three-Dimensional Matrix

Blood plasma containing human platelets were used to dilute human fibroblasts to a concentration of 5×10⁵ fibroblasts per milliliter of platelets. Three milliliters of the platelet mix was placed in each well of a twelve well cell culture plate. The tissue culture plate was pre-coated with fibrinogen (4.5 mg/ml) for 1 hour at 37° C. and then removed prior to addition of the mixture. Control wells contained platelets only without cells. Two hundred microliters of a thrombin solution (1000 U/ml in a 10% calcium chloride solution) was added to each well followed by a brief agitation of the well. This resulted in the formation of a three-dimensional matrix.

Approximately 2 milliliters of fibroblast growth media was then added on top of the hemostatic clot. The growth media used was DMEM with the following additives L-glutamine (4 mM), penicillin (100 U/ml), streptomycin (100 μg/ml), gentamycin (100 μg/ml), 10% fetal bovine serum (FBS), transferrin (5 μg/ml), insulin (5 μg/ml), amphotericin (0.25 μg/ml), and HEPES buffer (40 mM). The media contained concentrations of several growth factors including transforming growth factor beta (TGF-β 0.1-6 ng/ml), Vitamin C (vit C 25-100 μg/ml), and platelet derived growth factor AB (PDGF-AB 0.1-10 ng/ml). Concentrations of these growth factors were varied. Cultures were allowed to grow in a 37° C. incubator, in 95% air and 5% CO₂ atmosphere for 10 days. Media was changed every other day.

### EXAMPLE II

### Method of Producing Human Skin Equivalent

After incubating the matrix for ten days in fibroblast growth media with media changes on even days, human keratinocytes were plated on top of the matrices at a concentration of 3×10⁴ cells/cm². These matrices were overlayed with KGM supplemented with 10% FBS and allowed to grow submerged for 3 to 4 days, with 1 intervening media change. Concentrations of the above growth factors (PDGF, TGF-β and Vitamin C) were maintained. After 4 days the matrices were raised to create an air-liquid interface for periods of up to 4 weeks. A differentiation media was used during the airlifted phase consisting of a 3:1 mix of DMEM and HAM F-12. Additives to this media included 1-glutamine (2 mM), 10% FBS, human epidermal growth factor (0.2 ng/ml), Insulin (5 μg/ml), transferrin (5 μg/ml), tri-iodothyronine (2 μM), sodium pyruvate (1 mM), adenosine (1.8×10⁻⁴M), hydrocortisone (0.5 μg/ml), penicillin (100 U/ml), streptomycin (100 μg/ml), amphotericin (100 ng/ml).

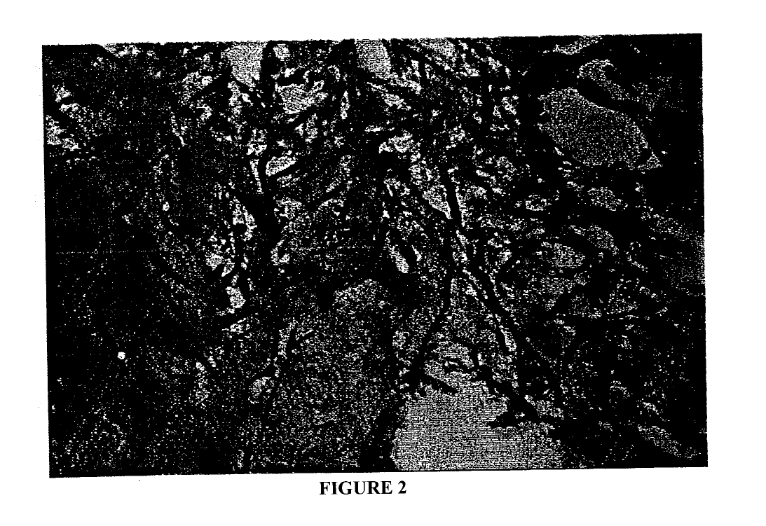

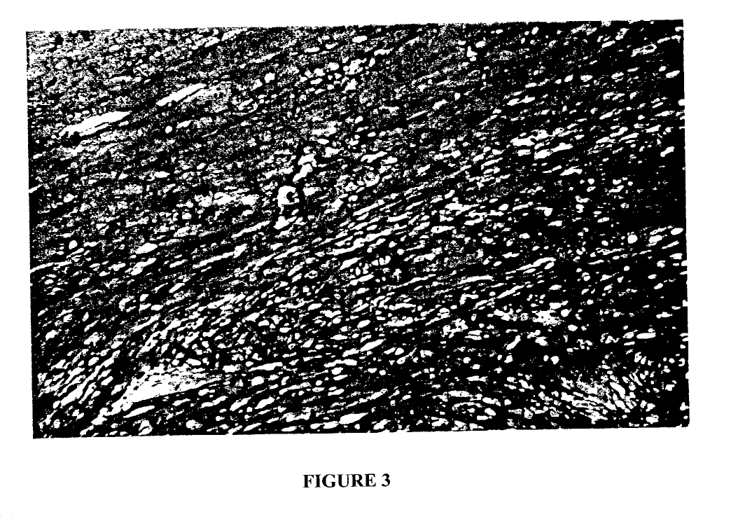

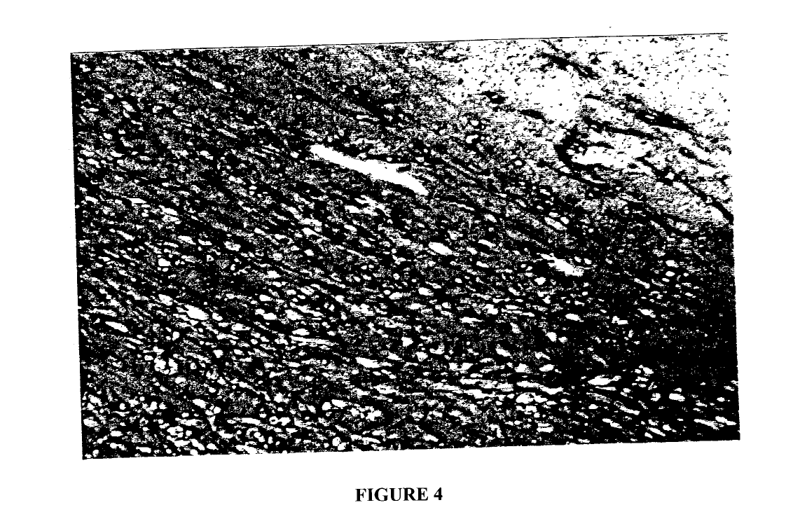

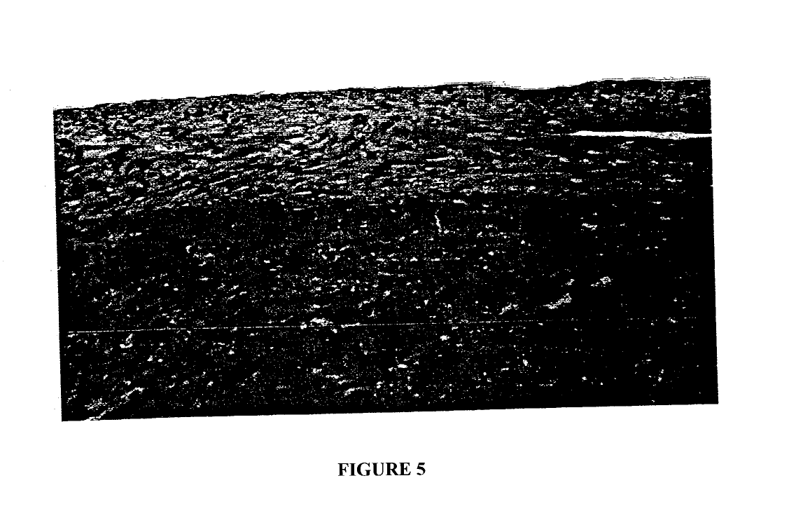

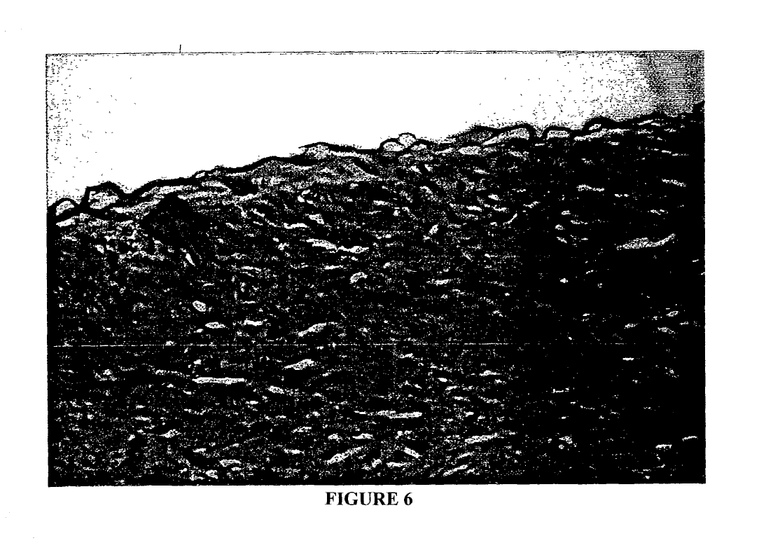

TGB-β was added to inhibit rapid cell division by the fibroblasts, thereby preventing destruction of the matrix. This was confirmed histologically as matrices maintained without TGB-β demonstrated excessive cellular multiplication. This effect was maximal at concentrations at or above 1 ng/ml of TGB-β. As shown in FIGS. 2 and 3, H&E stains revealed well-established fibroblasts within the matrices and a multi-stranded fibrous material surrounding the cells, most pronounced on the top of the matrix. Immunohistochemical staining showed these strands to be composed collagens I and III, as depicted in FIGS. 4 and 5, respectively. The majority of the collagen production was in the area of high cellular concentration. Matrices without fibroblasts did not stain for collagen. Properties of matrices harvested immediately after formation included stain localization to the fibroblasts themselves and no staining in the extra-cellular matrix. Quantitative analysis of collagen production revealed Vitamin C (ascorbic acid) as a significant stimulus for collagen production. PDGF appeared to enhance collagen type III production as did TGF-β. But TGF-β appeared to inhibit collagen type I production. The matrices that were airlifted and plated with keratinocytes developed a stratified epithelial layer with the underlying matrix populated with fibroblasts, as shown in FIGS. 6 and 7.

The matrices were harvested both from the standard wells and from the air-liquid interface wells on a weekly schedule. The matrices were then fixed, paraffin embedded, sectioned, and stained. Hematoxylin and eosin (H&E) stains and immunohistochemical stains for collagens I and III were performed. Sections stained for collagen were then digitized and analyzed using a digital image analysis software package, Image Pro 4 from Media Cybernetics (Silver Spring, Md.). Area of stain and integrated optical density were the image properties quantified in order to compare the effects of the various growth factors on collagen production. Table I shows the effect of TGF-β concentrations on collage production as quantified using digital image analysis, measuring both area and integrated optical density.

TABLE I
Effect of TGF-β Concentration on Collagen Production at 5 Weeks

| | | 0.1 ng/ml TGF-β | 1 ng/ml TGF-β | 2 ng/ml TGF-β | P |
|---|---|---|---|---|---|
| Collagen I | Area (px) | 18235 | 53845 | 202292 | <0.001 |
| | IOD | 3116879 | 9327031 | 1951400 | <0.01 |
| Collagen III | Area (px) | 36468 | 63998 | 127930 | <0.001 |
| | IOD | 6154005 | 1050433 | 1851266 | <0.001 |

### EXAMPLE III

### Method of Culturing Cells Using the Three-Dimensional Matrix

The present invention may also provide for a method of culturing cells in a three-dimensional matrix inoculated with fibroblasts from the tissue in which those cells are normally found. A three-dimensional matrix formed in this manner may simulate the in vivo cellular environment of the cells, thereby allowing the growth and differentiation of these cells.

The traditional monolayer cell culture techniques do not simulate the in vivo cellular environment and thus do not permit cells to fully differentiate. It is known that the extracellular matrix and cell-cell interaction influences the behavior and differentiation of cells. Cells inoculated on a three-dimensional matrix have been reported to penetrate the matrix and establish a tissue-like histology. For example, collagen gels have been utilized to culture mammary epithelial cells to form specialized structures. Yang, et al., Sustained growth in a primary culture of normal mammary epithelial cells embedded in collagen gels, Proc Nat Acad Sci USA 1980; 77(4):2088-92.

In one embodiment, hepatocytes, may be cultured using the three-dimensional collagen matrix of the present invention. Hepatocytes are anchorage-dependent, highly differentiated cells that are difficult to maintain in vitro. Conventional methods have attempted to culture hepatocytes in monolayers on plastic dishes. Bissell D M, et al., Parenchymal cells from adult rat liver in nonproliferating monolayer culture. I. Functional studies. J. Cell. Biol. 1973; 59:722-34. Hepatocytes in monolayer cell cultures have been found to de-differentiate, lose adult liver phenotype within a week of incubation, and eventually die off. On the other hand, it has been suggested that hepatocytes that are maintained in a culture closely resembling their in vivo environment are better capable of continuing to actively express specific cell functions. Clement B, et al., Long-term co-cultures of adult human hepatocytes with rat liver epithelial cells: modulation of albumin secretion and accumulation of extracellular material. Hepatology 1984 4(3):373-80.

For example, hepatocytes have been cultured by placing a suspension of hepatocytes between two layers of sterilized rat-tail collagen to simulate the in vivo cellular environment, as disclosed in U.S. Pat. No. 5,942,436. Accordingly, hepatocytes may be cultured in a three-dimensional matrix of the present invention, such that they could maintain their differentiated structure for extended period of time without loss of its functions. In preferred embodiments, the three-dimensional matrix may be inoculated with liver fibroblasts to produce collagen type I and may optionally be provided with other extracellular components to simulate the in vivo extracellular environment of the hepatocytes.

In another embodiment of the present invention, the three-dimensional collagen matrix of the present invention may be used to culture hematopoietic progenitors for the purpose of autologous bone marrow transplantation. Collagen gels culture techniques have been reported to be superior to the widely used agar or methylcellulose systems for the hematopoietic progenitors because of the equal or higher number of colonies detected, the easy phenotypical identification of colonies in stained gels, and the ability to store high-quality documentation. Dobo, I et al. Collagen matrix: an attractive alternative to agar and methylcellulose for the culture of hematopoetic progenitors in autologous transplantation products. J. Hematother 1995; 4(4):281-7.

The examples and embodiments described herein are for illustrative purposes only. Various modification or changes in light of the disclosure and claims that will be suggested to persons skilled in the art and disclosed in the cited references are incorporated herein by reference.

### EXAMPLE IV

### Biocompatible Three-Dimensional Scaffold for the Matrix

The three-dimensional matrix disclosed in this invention may be formed into a desired shape by casting it within a mold or by impregnanting a biocompatible scaffolding material. The biocompatible scaffolding material may be formed into a mesh, woven, braided, knitted or otherwise manipulated to form a desired shape.

The materials used to form the three-dimensional scaffolds on which the matrix is cast must be biocompatible. Also, these materials must allow for cell growth and for the retention of differentiated cell functions within the matrix. Furthermore, these materials must possess the physical characteristics, such as large surface to volume ratios, mechanical strength, and easy processing into complex shapes, such as those required for the formation of bone equivalents, as disclosed in U.S. Pat. No. 5,686,091.

In one embodiment, a number of biodegradable materials may be used to form the scaffold. Generally, any biodegradable polymer that produces nontoxic degradation products may be used as scaffolding material for tissue engineering. Peters, M C and Mooney, D J. Synthetic extracellular matrices to guide tissue formation. In: Ikada, Y, editor; Enomoto, S, co-editor. Tissue Engineering for Therapeutic Use 2, Amsterdam: Elsevier; 1998:55-65.

As disclosed in U.S. Pat. No. 6,140,039, biodegradable scaffold materials used for this purpose may include, but are not limited to, polyglycolic acid, polylactic acid, collagen, collagen sponges, cat gut sutures, cellulose, gelatin, dextran, and polyalkanoates. Other biodegradable scaffold materials are disclosed in U.S. Pat. No. 5,686,091, incorporated by reference as if fully set forth herein. Thus, the use of biodegradable materials may form a temporary scaffold within which a tissue can develop and is ultimately replaced entirely by cells and the natural extracellular matrix.

Non-biodegradable materials may also be used to form the desired tissue scaffold. These materials include, but are not limited to, metals, polyamides, polyesters, polystyrene, polypropylene, polyacrylates, polyvinyl compounds, polycarbonate, polytetrafuorethylene, thermanox, nitrocellulose, and cotton.

In another embodiment, the three-dimensional matrix of this present invention may impregnate a three-dimensional polymer foam, disclosed in U.S. Pat. No. 5,686,091. Accordingly, the polymers suitable for use in the practice of this invention include poly(lactic acid) (PLLA), poly(glycolic acid) (PGA), poly(lactide-co-glycolide) (PLGA), bisphenol-A based poly(phosphoester)s such as poly(bisphenol A-phenylphosphonate) (BPA/PP, poly(bisphenol A-ethylphosphate) (BPA/EOP), poly(bisphenol A-ethylphosphonate) (BPA/EP), poly(bisphenol A-phenylphosphonate) (BPA/POP), poly-bis(2-ethoxy)hydrophosphonic terephthalate (PPET), and copolymers of the BPA series and PPET. Accordingly, the three-dimensional scaffold may be sculpted to different sizes, shapes and forms to allow for the growth of the tissue cells.

Other examples of materials and construction methods for these scaffolds are provided by U.S. Pat. Nos. 5,863,984, 5,770,417, and 5,916,265. These patents are hereby incorporated by reference as if fully set forth herein.

The examples and embodiments described herein are for illustrative purposes only. Various modification or changes in light of the disclosure and claims that will be suggested to persons skilled in the art and disclosed in the cited references are incorporated herein by reference.

### EXAMPLE V

### Method of Producing Blood Vessel Equivalents

Generally, blood vessels share a common basic structure. The various types of blood vessels, such as the arteries, veins, and capillaries, are distinguished from each other on the basis of the thickness of the vascular wall and differences in the composition of each respective layer. The three layers of the blood vessel, starting from the lumen outward, are the tunica intima, the tunica media and the tunica adventitia. The tunica intima is the inner lining of the blood vessel, which consists of a single layer of endothelial cells (endothelium). Next is the tunica media, the muscular middle layer, which consists primarily of circumferentially arranged layers of smooth muscle cells. Last is the outer supporting tissue layer, the tunica adventitia, which consists mostly of longitudinally arranged collagenous tissue and elastic fibers. Wheater P R. et al. Wheater 's Functional Histology: A Text and Colour Atlas. Edinburgh: Churchill Livingstone; 1993.

Accordingly, a blood vessel equivalent may be produced as one embodiment of this invention by methods known in the prior art.

For example, the methods of producing human blood vessel equivalents disclosed in U.S. Pat. Nos. 4,485,096, 4,539,516, 4,539,716, 4,546,500, 4,837,379, 5,256,418, and 6,140,039 are generally applicable to the methods of the present invention and are hereby incorporated by reference as if fully set forth herein.

In one embodiment, a blood vessel equivalent may be formed by casting various layers of the hemostatic clot mixture of this present invention containing cultured vascular cells specific to each layer sequentially around a suitable rod or mandrel. First, a hemostatic clot mixture containing arterial smooth muscle cells may be cast around a cylindrical member. The cylindrical member may be a rod, a circle cylinder or a tube. The smooth muscle cells contribute collagen, elastin, and other molecules to the matrix. Other extracellular components may optionally be added, such as hyaluronic acid. Next, an outer layer of adventitious fibroblasts may be cast on top of the first layer. Last, the internal surface of the resulting vessel may then be lined with a layer of endothelial cells to form a complete blood vessel equivalent. A mesh sleeve may be cast around the outer surface of or embedded within the smooth muscle layer to reinforce the blood vessel equivalent. U.S. Pat. No. 4,546,500.

It may be desirable to line the internal surface with specialized cells having a predetermined therapeutic value, such as pancreatic β cells to boost insulin supply in the blood. Alternatively, cultured cells from patients with a particular disease may be incorporated into the blood vessel tissue equivalent for the study of certain diseases. U.S. Pat. Nos. 4,485,096, 4,539,516, 5,256,418.

The method of producing a blood vessel equivalent disclosed above may also be achieved in a casting chamber to a defined diameter and size. The casting chamber may be in form of a cylinder with an axially centered rod having a diameter of the desired blood vessel equivalent, as disclosed in U.S. Pat. Nos. 4,539,716 and 4,546,500.

Furthermore, in yet another embodiment, blood vessel equivalents in accordance with the present invention may be produced by combining cells isolated from each layer of the patient's arteries in the hemostatic clot mixture and incorporating them on separate synthetic meshes. After an appropriate degree of confluence is reached, a layer of endothelial cells may be seeded on top of the media layer of the blood vessel equivalent. In accordance with the disclosure in U.S. Pat. No. 6,140,039, a biocompatible mesh may be used to form vessels of defined dimensions. The mesh may be in form of rectangular strips having a width that equals the circumference of the blood vessel equivalent and having any desired length. The blood vessel equivalent may be formed by joining the long edges together. In one embodiment, the mesh may also be in form of a tubular shape of varying diameters such that they may be combined.

Specifically, a first mesh may be impregnated with a first hemostatic clot mixture containing fibroblasts isolated from the adventitia of the patient's blood vessel. A second mesh may be impregnated with a second hemostatic clot mixture containing cells isolated from the intima or media of the patient patient's blood vessel. These cells may include elastin-producing smooth muscle cells and fibroblasts. After the elastin-producing cells have proliferated to the appropriate extent, the first and second mesh may be combined. The smooth muscle cells will continue to proliferate and produce elastin in an environment that simulates the environment of the natural arterial walls. Endothelial cells isolated from the blood vessel of the same patient may then be seeded on top of the second mesh-matrix layer and incubated until a confluent layer is formed to produce a complete blood vessel equivalent. U.S. Pat. No. 6,140,039.

### EXAMPLE VI

### Method of Producing Bone Tissue Equivalents

Bone is a specialized form of connective tissue that, like other connective tissues, consists of cells and an extracellular matrix. The feature that distinguishes bone from other connective tissues is the mineralization of the matrix. This produces an extremely hard tissue capable of providing support and protection. Bone matrix consists of type I collagen and a ground substance containing proteoglycans and noncollagenous glycoproteins. Both the collagen and ground substance components are mineralized. Ross, M. H. et al. Histology. Baltimore: Williams & Williams; 1995.

Bone tissue engineering requires a successful and productive interaction between osteoprogenitor or osteoblastic cells, osteoinductive growth factors, and an osteoconductive matrix. The osteoblastic cells can be derived from the host tissue. These cells are directly responsible for forming the newly engineered bone tissue. The osteoinductive growth factors can also be secreted by transplanted cells or delivered from an outside source. They induce the proper recruitment, differentiation, and proliferation of the appropriate cell types. The osteoconductive matrix can be used to provide mechanical support and thus serve as a scaffold for tissue as well as serving as a vehicle for transplanted cells or other growth factors.

Hence, bone tissue equivalents may be used to provide artificial hip joints, knee joins, and finger joints, and maxillofacial implants, such as dental implants. It can also be used for special surgery devices, such as spacers, or bone fillers for use in augmenting or reconstituting damaged or lost bone. The bone formation can be optimized by variation in mineralization and a provision of a load-bearing scaffold coated with the matrix of this present invention and further with a bone filler comprising a matrix.

A wide variety of materials have been investigated and proposed for use in bone defects, including, but not limited to, metals such as titanium fiber, ceramics such as hydrooxyapatite or tricalcium phosphate, polymers, polymer/ceramic composites, and even marine coral. Bancroft, G N and Mikos, A G. Bone tissue engineering by cell transplantation. In: Ikada, Y, editor; Enomoto, S, co-editor. Tissue Engineering for Therapeutic Use 5, Amsterdam: Elsevier; 200:151-64. Biodegradable and bioresorbale three-dimensional scaffolding materials for the repair and replacement of diseased or injured bones have also been disclosed in U.S. Pat. No. 5,522,895, herein incorporated by reference.

In one embodiment, bone tissue equivalents may be prepared by incorporating demineralized bone powder into the three-dimensional collagen matrix of this present invention. The interactions between demineralized bone powder implanted subdermally have been previously described. This embodiment allows the bone tissue equivalent to be cast into any shape according to the mold into which the materials are cast, as the fibroblasts cells transform the demineralized bone powder to bone. The demineralized bone powder may be prepared by acid extraction methods known to those skilled in the art. The demineralized bone powder may be sterilized in 70% ethanol, washed and soaked overnight in 1X McCoy's 5A medium to saturate it with the medium. A three-dimensional matrix may be cast in a mold of any configuration and size to form the final bone equivalent. In this embodiment, the bone equivalent may be formed by inoculating the matrix with fibroblasts, serum, insulin, testosterone and somatomedin and adding the de-mineralized bone powder. Optionally, a sheet of stainless steel mesh of any shape may be used to maintain the shape of the bone equivalent. U.S. Pat. No. 4,485,097.

In another embodiment, the matrix of this present invention may be used in the construction bone tissue equivalents as disclosed in U.S. Pat. No. 6,299,650 by applying undifferentiated cells on a substrate discussed above, and directly contacting the undifferentiated cells within the matrix for a sufficient time to differentiate and form osteoblasts, wherein the ostoblasts produce an extracellular bone tissue matrix. Preferably, autologous cells are used in the construction of the bone tissue equivalent.

The examples and embodiments described herein are for illustrative purposes only. Various modification or changes in light of the disclosure and claims that will be suggested to persons skilled in the art and disclosed in the cited references are incorporated herein by reference.

### EXAMPLE VII

### Method for Producing Cartilage Tissue Equivalents

Cartilage is an avascular tissue that consists of a population of chondrocytes embedded within a highly organized extracellular matrix. The chondrocytes are responsible for the synthesis and maintenance of the macromolecules of the matrix. The major components of the cartilage matrix are collagen type II and proteoglycans. In cartilage, collagen forms a dense meshwork of fibers which provides structural shape and form to the tissue.

Accordingly, in one embodiment of this invention, a cartilage tissue equivalent may be produced by including chondrocytes in the three-dimensional collagen matrix, allowing their synthesis of a new cartilage matrix. The cartilage tissue equivalent may also be produced by including cells that elaborate cartilage-specific macromolecules and extracellular matrix proteins in the three-dimensional collagen matrix. These cells may include chondrocytes, chondrocytoprogenitors, fibroblasts, fibroblast-like cells and /or cells capable of producing collagen type II and other collagen types, and proteoglycans which are typically produced in cartilaginous tissues. U.S. Pat. No. 5,902,741. The cartilage tissue equivalent may be formed into a desired shape by impregnating it on a suitable scaffolding material.

Studies have shown that cultured homologous embryonic chondrocytes embedded in a biologically resorbable gel placed in a surgically created defect on the joint surface can repair the injury for up to 18 months. Itay, S, et al. Use of cultured embyonal chick epiphyseal chondrocytes as grafts for defects in chick articular cartilage. 1987; Clin. Orthop. 220:284-303. It has also been shown that the mechanical environment around cartilage has a significant effect on the metabolic rate of chondrocytes and the biochemical composition of the tissue. For example, cartilage allografts cultured under cyclic load exhibited superior histological and biochemical properties when compared to tissues, which were cultured but not loaded. Accordingly, mechanical or shear force may be applied to the chondrocyte matrix to enhance the maintenance of the chondrocyte phenotype to that found in vivo. U.S. Pat. No. 5,928,945.

Other embodiments are disclosed in U.S. Pat. Nos. 6,171,340 and 6,197,061 and are hereby incorporated by reference as if fully set forth herein. The examples and embodiments described herein are for illustrative purposes only. Various modification or changes in light of the disclosure and claims that will be suggested to persons skilled in the art and disclosed in the cited references are incorporated herein by reference.

### EXAMPLE VIII

### Tests for Determing the Interation of a Living Tissue Equivalent with Various Agents

The present invention may also provide for methods of determining the interaction of tissue with at least one agent through the use of tissue equivalents, as disclosed in U.S. Pat. No. 4,835,102. The results of tests performed on living tissue equivalents should reflect the response of the natural tissue more closely than a corresponding test conducted with animal or cadaver tissue. Furthermore, the use of living tissue equivalents should obviate the need for human or animal subjects for the study of the effects of various harmful pharmacological, chemical, biological, synthetic, and other agents on the selected tissue.

In one embodiment of this present invention, apparatus, methods, and kits based on tissue equivalents may be provided by the present invention for use in determining the interaction of a selected tissue and selected agents, including, but not limited to, (1) the measurement of the rate and extent of penetration of agent(s) through a tissue equivalent, (2) the interaction of agent(s) reflected by changes in cell permeability in the tissue equivalent, (3) the responses of tissue equivalent cells to agent(s) that provoke the release of various regulatory or signaling molecules into the intracellular tissue fluids, and (4) the responses of tissue equivalent cells together with specialized immune cells to agent(s) that are considered allergens. U.S. Pat. No. 4,835,102

For example, the interaction between the tissue equivalent and the agent(s) may be determined by analyzing the tissue equivalent itself, the intracellular fluid of the tissue equivalent, or the solution containing the particular agent(s). Test may then be conducted to detect and quantify the amount of agent(s) absorbed in the tissue by methods known to those skilled in the art. U.S. Pat. No. 4,835,102.

Accordingly, in one embodiment, the kinetics of tissue penetration by selected agent(s) in a tissue equivalent may be measured by placing the tissue equivalent in a chamber and subjecting it to a solution containing the agent(s). This solution may be pumped or fed through the chamber under conditions to simulate blood flow as a function of time. Data may be obtained to calculate the permeability constant or other relevant parameters, such as the time for initial penetration or the percentage of the agent absorbed by the tissue equivalent from analyzing the tissue equivalent itself, the intracellular fluid of the tissue equivalent, or the solution containing the agent(s).

In another embodiment, the changes in tissue permeability may be determined by the interaction of the tissue equivalent and the agent(s) by measuring the release of intracellular proteins and other chemicals as a result of tissue cell damage from exposure to the particular agent(s). The release of cellular proteins may then be measured by methods well known to those skilled in the art, including, but not limited to radioimmunoassays, enzyme immunoassays, enzyme-linked immunoassays, and fluorescent immunoassays. Other chemicals released by the tissue equivalent cells in response to physiological stress as a result of the agent(s) may be also measured by methods known to those skilled in the art.

In yet another embodiment, the tissue equivalents may be used to measure responses to various agents that emanate from immune cells incorporated into the tissue equivalent. For example, the epidermis of the skin contains Langerhans cells of the skin, which act as antigen-presenting cells in the skin. When stimulated, they migrate through the dermal lymphatics to the paracortical zones of the regional lymph nodes for presentation of antigen to T lymphocytes. These Langerhans cells provide the first step in the immune reaction of the skin. Accordingly, a skin immunoreactivity test may be performed by exposing skin equivalents to the agent(s) and then by measuring the migration of activated Langerhans cells out of the epidermis of the skin equivalent by immunofluorescence or other methods known to those skilled in the art.

In another example, macrophages may included in the tissue equivalent to provide another source of the assayable lymphokine, IL-1, secreted by the cells in response to substances that initiate humoral immune reactions. In yet another example, sensitized T cells responsive to particular classes of immunogens, together with mast cells that release strong signals such as histamine, may be incorporated in the skin tissue equivalent to provide immune signals. These immune signals may be easily assayed because of their degree of amplification. The examples and embodiments described herein are for illustrative purposes only. Various modification or changes in light of the disclosure and claims that will be suggested to persons skilled in the art and disclosed in the cited references are incorporated herein by reference.

### EXAMPLE IX

### Assay to Study the Pathology of Diseased Tissues and the Efficiacy of Treatment

In yet embodiment of the present invention, the three-dimensional matrix may be inoculated by a variety of tumor cells to study the mechanisms of tumor growth and metastasis and to further study the efficacy of chemotherapeutic agents on both tumor and normal cells. To this end, tumor cells derived from the biopsy sample of a patient may be included in the three-dimensional collagen matrix or into a living tissue equivalent formed therefrom to provide a model of the diseased tissue. The tumor model may then be used to create an effective and individualized chemotherapy treatment for the patient that selectively targets malignant and diseased cells.

The examples and embodiments described herein are for illustrative purposes only. Various modification or changes in light of the disclosure and claims that will be suggested to persons skilled in the art and disclosed in the cited references are incorporated herein by reference.

### EXAMPLE X

### Use of Tissue Derived from the Three-Dimensional Matrix in Tissue Microarrays

A tissue equivalent derived from the three-dimensional matrix may be used in a tissue microarray. Histological sections, including paraffin-embedded and frozen sections, may be prepared from tissue equivalents.

Tissue microarrays provide a method for high-throughput molecular profiling of tissue specimens (Kononen et al., Nature Medicine, 4: 844-847, 1998). The technology has been enhanced by the development of instruments specifically designed to construct the tissue arrays. One such instrument is the ATA-27 Automated Tissue Arrayer (Beecher Instruments, Silver Spring, Md.), as described in U.S. Pat. No. 6,103,518.

High-throughput genome screening technologies, such as cDNA microarrays, and serial analysis of gene expression (SAGE) have made it possible to survey thousands of genes at a time from tissue and cell preparations. The translation of such information to improved diagnostic, prognostic and therapeutic applications in the clinic requires extensive time-consuming validation, prioritization and extension of protein expression information. For example, hundreds or even thousands of clinical specimens are required to ascertain the significance of a new diagnostic test or therapeutic target. This is a tedious process and the use of conventional molecular pathology techniques combined with the relative scarcity and unreliability of replicate identical tissue sources has stymied development in this exciting new area of research.

The use of tissue microarrays has helped overcome these hurdles and has contributed to facilitating such “genome-scale” research. This technology enables high-throughput molecular analyses of hundreds of tissue specimens or cells in a single experiment. Tissue microarrays are constructed by acquiring cylindrical biopsies from individual tissues into a tissue microarray block, which is then sliced into many sections for probing DNA, RNA or protein targets. A single immuno-staining or in situ hybridization reaction may provide information on all of the specimens on the slide, while subsequent replicate sections can be analyzed with other probes or antibodies. Construction of multiple replicate blocks may allow up to thousands of sections to be generated from the same series of tissue specimens.

Tissue microarrays provide a means for rapid, large-scale molecular analysis of thousands of tissue specimens with thousands of probes for various DNA, RNA and protein targets. For example, cDNA and tissue microarray technologies may be used in combination to uncover genes involved in breast and prostate cancer progression. Taken together, the cDNA and tissue microarrays provide a powerful approach for the in vivo validation of gene discoveries, as well as a means to rapidly assess the clinical significance of molecular alterations in cancer and other diseases.

Samples of the original tissues for arraying are acquired from morphologically representative regions of regular formalin-fixed paraffin-embedded or frozen tissue blocks. Core tissue biopsies are taken from individual “donor” blocks and arrayed into a new “recipient” paraffin block using a tissue microarraying instrument, such as the ATA-27 Automatd Tissue Arrayer (Beecher Instruments, Silver Spring, Md.). Typically, 0.6 mm cylinders are utilized because they convey histological information, allow up to 1000 specimens to be arrayed in each block, yet result in little damage to the original blocks. This sample size may be adjusted, however, to accommodate alternative uses. The donor block is manually positioned for sampling based on a visual alignment with the corresponding HE-stained section on a slide. The region of interest for sample acquisition from each block is carefully selected from the H&E stained slide.

After the block construction is completed, 5-8 μm sections of the resulting tissue microarray block are cut with a microtome. An adhesive-coated tape sectioning system, such as the Paraffin-Tape Transfer System (Instrumedics, Hackensack, N.J.), is useful for assisting sectioning of the array blocks. On average, 200 sections can be cut from one tissue microarray block. HE-staining for histology verification may be performed every 50th section cut from the block.

The tissue microarray slide may be evaluated either manually or utilizing a high-throughput digital imaging system. Brightfield images may be acquired using a high-resolution (4 k×3 k pixels) digital camera, such as those produced by Carl Zeiss (Carl Zeiss Microimaging, Inc., Thomwood, N.Y.).

One method of preparing a tissue equivalent for use in a tissue microarray may be paraffin embedding. The tissue equivalent may be fixed in formalin, washed and dehydrated in a series of alcohol solutions containing an increasing amount of alcohol. The alcohol may then be removed by washing the tissue equivalent in an organic solvent, such as xylol or toluol, followed by paraffin infiltration of the sample. Once cooled and hardened, the paraffin may be trimmed to an appropriate-sized block.

Another method to prepare a tissue equivalent for use in a tissue microarray may be through frozen section. The tissue equivalent may be snap-frozen in liquid nitrogen. For example, a blockholder may be placed on a snap-freezing device. Embedding media, such as Cryo-Gel (Instrumedics, Hackensack, N.J.), may be dispensed onto the blockholder and a tissue specimen positioned on top. A chilled heat extractor, upon contacting the tissue equivalent, may snap-freeze the sample into a block.

Small tissue core biopsies may be punched from selected regions of the paraffin-embedded or frozen blocks using a thin-wall stainless steel tube sharpened like a cork borer. For paraffin-embedded samples, hemotoxin and eosin (HE)-stained sections overlayed on the surface of the donor blocks may guide sampling from morphologically representative sites in the tissues. A solid stainless steel stylet may transfer the tissue cores into defined array coordinates in the recipient block. An adhesive-coated tape sectioning system may assist in cutting the tissue microarray block. Each tissue element in the array may have a defined diameter and defined spacing between array elements. These microarrays may then be used in applications that include, but are not limited to, those that for probe for DNA, RNA, protein, lipid, and other intracellular or extracellular molecular targets. In addition, the microarray sections may be utilized for microscopic morphological analysis.

### EXAMPLE XI

### Method of Genetically Modifying Cells or Living Tissue Equivalents Grown or Derived from the Three-Dimensional Matrix Using a Cationic Lipid Complex

The present invention may also provide for a method of growing genetically modified cells or tissues in a three-dimensional matrix. Genetically modified cells or tissues may produce intracellular or extracellular proteins that modify the cell's structure or function. Moreover, such manipulation may alter diagnostic, and/or morphological characteristics of the cell or tissue. Alternatively, genetically modified cells in a tissue equivalent may be used to secrete proteins bearing cell or tissue-specific modifications (i.e. cell-type specific glycosylation patterns, etc.). These proteins may then be isolated in large quantities and used for a variety of purposes. These tissue and cell-specific modifications may be superior to current methods of expression. Current methods of protein expression include, but are not limited to, using prokaryotic systems, baculovirus systems, Sindbis systems, CHO cell systems, plant systems and genetically altered cells that are implanted into animals, which then produce the desired protein.

In one embodiment, cells that will be grown in the three-dimensional matrix may be transfected using a cationic lipid complex, such as Lipofectamine (Invitrogen Corp., Carlsbad, Calif.). The day before transfection, the cells may be trypsinized, counted and plated such that they are 90% confluent on the day of transfection. The cells may then be plated in 0.5 ml of normal growth medium containing serum and antibiotics. For each well of cells, DNA may be diluted to between 0.8 μg and 1.0 μg into 50 μl of medium without serum. For each well of cells, 1 μl to 3 μl of cationic lipid reagent (“reagent”) may be diluted into 50 μl of growth medium. Once diluted, the reagent may be combined with the diluted DNA within 30 min.

Next, the diluted DNA may be mixed with the diluted reagent and incubated for 20 minutes at room temperature. The cell growth medium may be replaced with 0.5 ml of serum-free medium and the DNA-reagent complexes added to the well, followed by gentle rocking to ensure thorough mixing. The cells may be incubated at 37° C. in humidied CO₂ for 24 to 48 hours.

After either 24 to 72 hours for transient transfection, or 2 to 3 weeks for stable transfection, the cells may be asssayed to determine which of them express the gene of interest. Those cells expressing the gene of interest may be isolated and used to inoculate the three-dimensional matrix for further growth and utilization.

### EXAMPLE XII

### Method of Genetically Modifying Cells or Living Tissue Equivalents Grown or Derived from the Three-Dimensional Matrix Using Calcium Phosphate Coprecipitation

In another embodiment, the cells may be genetically modified using a calcium phosphate co-precipitation technique. On day one, cells may be plated in 60 mm dishes at a density of 10⁶ cells/ml. On day 2, following overnight incubation at 37° C. in humidified CO₂, the media may be replaced with fresh media 3 to 4 hours prior to transfection.

The transfection mixture may be created by the drop-wise addition of a mixture “A” comprised of 18 μl of 2M CaCl₂ and 10 μg of DNA in a total volume of 150 μl of sterile H₂0, into 150 μl of a second mixture comprised of 2X Hepes Buffered Saline (HBS). Air may be bubbled through the HBS mixture during the drop-wise addition of Mixture A to. This process may take place over a 1-2 minutes and until Mixture A is depleted.

A fine precipitate may form and may be incubated at room temperature for 30 minutes. The precipitate may be added drop-wise to the media in the 60 mm dish containing the cellsand incubated overnight at 37° C. in a humidified CO₂ incubator.

On day three, the media may be removed from the cells, and the cells washed twice with 1X Phosphate Buffered Saline (PBS) and, depending on the cell type, subjected to a glycerol or dimethylsulfoxide (DMSO) shock.

To subject the cells to glycerol shock, a 15% glycerol shock solution may be prepared in 1X HBS. The cells may be washed once in 1X PBS, followed by the addition of 2 ml of the 15% glycerol solution to each 60 mm dish and then incubated at room temperature for 2.5 minutes. The glycerol solution may be removed; the cells may be washed in a 1X PBS solution followed by replacement of the 1X PBS solution with fresh media.

Alternatively, to subject the cells to DMSO shock, the media may be removed from the 60 mm dish containing the cells and replaced with 2 ml of 1X PBS containing 10% DMSO, followed by incubation at room temperature for 2.5 minutes. The 10% DMSO solution may then be removed and replaced with fresh media.

After subjecting the cells to either glycerol or DMSO shock, the cells, once in fresh media, may be incubated for 24-48 hours at 37° C. in a humidified CO₂ incubator. Using DNA in the transfection process that, in addition to containing the gene of interest, also contains a selection marker (such as the neomycin resistance gene) may permit cells that express the gene of interest to be isolated by growing the transfected cells in a medium containing a selection agent. Examples of selection agents suitable for this purpose include, but are not limited to, neomycin, zeocin, blasticidin, hygromycin B and bleomycin. The isolated cells expressing the gene of interest may be used to inoculate the three-dimensional matrix creating a genetically manipulated tissue equivalent or cell culture.

### EXAMPLE XIII

### Method of Genetically Modifying Cells or Living Tissue Equivalents Grown or Derived from the Three-Dimensional Matrix Using Electroporation

In one embodiment, the cells may be genetically modified using electroporation. Cells, such as primary human peripheral blood lymphocytes, may be grown in 175 cm² flasks containing growth medium consisting of RPMI 1640 (Invitrogen Corp., Carlsbad, Calif.) supplemented with 20% fetal calf serum, 100 U/ml penicillin, 100 μg/ml streptomycin, 2 mM glutamine, 1% phytohemagglutinin (PHA) and 30 U/ml recombinant IL-2 (“complete medium”). The cells may be incubated at 37° C. in humidified CO₂ until they reach a concentration of approximately 2×10⁷ cells/ml. The cells may then be subjected to centrifugation at 400 g for 5 minutes, forming a cell-pellet at the bottom of the tube. The supernatant may be aspirated and the cells re-suspended in ice-cold growth medium at a concentration of 2×10⁷ cells/ml.

A volume of 200 μl of cells may be placed into an electroporation cuvette, which may have a 2 mm gap, to which 20 μg of DNA containing the gene of interest and a selection marker (such as a neomycin resistance gene), may be added and the mixture incubated on ice for 10 minutes. The cuvette containing the mixture may be placed in an electroporation device, such as an Electro Cell Manipulator™ (BTX, San Diego, Calif.) and may be subjected to a 45-50 msec pulse of 126V and may have an estimated field strength of approximately 0.63 kV/cm. The electroporation cuvette containing the mixture may then be removed from the electroporation device and incubated on ice for 10 minutes.

The mixture may then be placed into 10 ml of room temperature complete medium and incubated at 37° C. and humidified CO₂ for 24-72 hours. The cells containing the gene of interest may then be isolated by including a selection agent (such as neomycin) in the growth medium. These isolated cells containing the gene of interest may then be added to, and grown in, the three-dimensional matrix.

### EXAMPLE XIV

### Method of Genetically Modifying Cells or Living Tissue Equivalents Grown or Derived from the Three-Dimensional Matrix Using DEAE-Dextran

In one embodiment, the cells may be transfected using the DEAE-Dextran technique. Cells may be cultured in 24 well plates at a density of 10⁶ cells/ml the day before transfection. On the day of transfection, the cells may be harvested by being subjected to a trypsin/EDTA solutionand washed with plain RPMI 1640 (with glutamine) (Invitrogen Corp., Carlsbad, Calif.) one time and resuspended to a concentration of 10⁷ cells/ml in RPMI 1640 (with glutamine), containing DEAE Dextran (50-250 μg/ml final). Approximately 10 μl of 1 μg/μl DNA, comprising the gene of interest and a selection marker (such as a neomycin resistance gene), may be placed into round bottom tissue culture tubes, to which the DEAE Dextran cell suspension may be added. This mixture may be incubated for a total of 90 minutes at 37° C. in humidified CO₂, during which time the mixture may be agitated at 30 minutes and 60 minutes. DMSO may then be added at a final concentration of 10% and incubated at room temperature for 2-3 minutes, which may be followed by the addition of 9 ml of RPMI 1640 medium (without serum). The cells may then be subjected to centrifugation at 400 g for 5-10 minutes, after which the supernatant may be aspirated and the cells may be resuspended in 8 ml of complete medium.

The cells may then be seeded into 4 wells of a 24 well plate and incubated at 37° C. in humidified CO₂ overnight, after which the medium may be replaced with fresh medium and the cells incubated for an additional 24-72 hours. The cells may then be subjected to a trypsin/EDTA solution to dislodge the cells from the plate, followed by resuspension in complete medium to a concentration of 10⁶ cells/ml. The medium may contain a selection agent (such as neomycin) toxic to cells lacking the selection marker and may permit the isolation of cells containing the gene of interest. These genetically modified cells may then be grown in the three-dimensional matrix as either a genetically engineered cell culture line or as part, or all, of a tissue.

### EXAMPLE XV

### Method of Genetically Modifying Cells or Living Tissue Equivalents Grown or Derived from the Three-Dimensional Matrix Using Viral Infection

In one embodiment, the cells may be genetically modified by direct infection with a recombinant virus that carries the gene of interest. A number of viruses have been defined that can successfully infect and insert DNA into a cell's genomic DNA. Such a process may genetically manipulate the cell such that it will produce a non-native protein, or produce a native protein at higher levels.

In one embodiment, human liver cells may be infected with an adenovirus that has had a portion of the E1a and E1b genes replaced with a gene encoding for human LDL receptor and a selection marker (such as a neomycin resistance gene). A DNA element known to enhance the transcription of cis-linked genes, such as a CMV promoter, may be linked upstream of the gene of interest to promote enhanced transcription of the gene once inside the cell. By growing the cells in a medium containing a selection agent (such as neomycin), selection of successfully infected cells that express the virally introduced genes may be possible. Those successfully infected cells that express the gene of interest may be implanted into a human liver tissue equivalent in the three-dimensional matrix. If the tissue equivalent were then implanted into a patient, it may be more efficient at removing serum cholesterol as a result of the increased number of LDL receptors on the surface of the hepatocytes.

### EXAMPLE XVI

### Method of Genetically Modifying Cells or Living Tissue Equivalents Grown or Derived from the Three-Dimensional Matrix Using Microinjection

In one embodiment, cells to be grown in the three-dimensional matrix may be genetically modified by microinjection of DNA. Cells may be grown on 25 mm diameter coverslips placed in 6-well dishes. Coverslips may be placed in coverslip chambers containing 1 ml of medium supplemented with fetal bovine serum. A region of approximately 1 mm² on the bottom of the coverslip may be marked for subsequent microinjection on the stage of a fluorescence microscope resting on a bench top vibration isolation system fitted with an Eppendorf microinjection unit (Brinkmann, Westbury, N.Y.). Borosilicate microinjection pipettes may be pulled using a Sutter Instrument Flaming/Brown micropipette puller (Sutter Instrument Company, Novato, Calif.). The cells in the marked area may be microinjected with a solution containing DNA, which may have the gene of interest and may have a selection marker (such as the neomycin resistance gene). The medium may then be changed to medium containing 10% fetal bovine serum. The cells may be incubated at 37° C. and humidified CO₂. Cells containing the gene of interest and selection marker may be isolated by growing the cells in a medium that contains a selection agent (such as neomycin) and implanted into the three-dimensional matrix.

### EXAMPLE XVII

### Method of Genetically Modifying Cells or Living Tissue Equivalents Grown or Derived from the Three-Dimensional Matrix Using Biolistic Particle Delivery

In one embodiment, the cells may be genetically manipulated by the introduction of DNA using a biolistic particle delivery system, such as the PDS-1000/He Particle Delivery System (Bio Rad, Hercules, Calif.). A biolistic particle delivery system may use sub-cellular sized microprojectiles coated with DNA (or other biological material), accelerated at a velocity sufficient to penetrate celluluar membranes and matrices such that DNA is inserted into the the cell.

In one embodiment, the microprojectiles may be microscopic gold beads (“microcarriers”). A suspension of gold particles may be prepared by resuspending 60 mg of gold particles in 1 ml of 100% ethanol, which may be followed by subjecting the suspension to sonication, using a fine probe, twice for 30 seconds. The suspension may then be subjected to centrifugation at 14,000 g for 1 minute, forming a pellet at the bottom of the tube. The supernatant may be aspirated and the process of resuspending the gold beads in 100% ethanol, and subjecting them to sonication followed by centrifugation may be repeated twice. The pelleted gold beads may then be resuspended in 1 ml of sterile H₂0.

The DNA containing the gene of interest may also contain a selection marker (such as a neomycin resistance gene) and may be diluted to 1 mg/ml.

A gold-DNA suspension may be prepared by placing 5 μl of 1 mg/ml DNA into a microcentrifuge tube, followed by the addition of 50 μl of the gold suspension. The mixture may be subjected to a vortex for one minute at maximum speed on a bench-top vortex device. To the mixture, 50 μl of 2.5M CaCl₂ and 20 μl of 100 mM Spermidine (Amersham, Biosciences, Piscataway, N.J.) may be added, which may be followed by subjecting the mixture to a vortex. The mixture may be subjected to centrifugation at 10,000 g for 10 seconds and the supernatant may be discarded. The pellet may be resuspended in 60 μl 100% ethanol.

Macrocarriers may be sterilized by immersion in 95% ethanol, followed by air-drying. The center of the macrocarriers may be loaded with 10 μl the gold-DNA suspension microcarriers and air-dried. The biolostic delivery apparatus (“gun”) may be prepared by wiping the chamber, holders and stages with 70% ethanol and may be followed by air-drying. The rupture disk may be soaked in 70% ethanol, placed in the rupture disc holder and screwed tightly into the gas acceleration tube after the helium gas, vacuum pump and power to the gun are turned on. The macrocarrier launch plate may be assembled by placing a piece of stopping screen in front of the macrocarrier. The macrocarrier bearing the DNA-coated microcarrier may be face down to the screen. The macrocarrier cover lid may be screwed on tightly, the launch assembly plate may be placed in the chamber and the chamber door may be closed. The gun may be fired to deliver the DNA-coated microcarrier particles to the cells. The chamber may be opened the tissue sample removed.

Growing the cells in a medium containing a selection agent (such as neomycin) may select for the cells that have successfully incorporated the DNA into their genome. The cells and/or tissues may be directly implanted into the three-dimensional matrix.

What is claimed is:

1. A three-dimensional matrix comprising a fibrin matrix and fibroblasts.

2. The three-dimensional matrix of claim 1 wherein the fibrin matrix comprises fibrinogens and means for polymerizing and cross-linking the fibrinogens.

3. The three-dimensional matrix of claim 1 wherein the fibrin matrix comprises fibrinogens and a thrombin.

4. The three-dimensional matrix of claim 1 wherein the fibrin matrix comprises a blood plasma and a thrombin.

5. The three-dimensional matrix of claim 1 further comprising one factor selected from the group consisting of a platelet, a factor XIII, a clotting factor, a fibronectin, a non-Willebrand factor, and a growth factor.

6. The three-dimensional matrix of claim 5 wherein the growth factor is selected the group consisting of TGF-β, PDGF, FGF, EGF, VEGF, IL-1, TNF and vitamin C.

7. The three-dimensional matrix of claim 1 further comprising a means for accelerating the formation of the three-dimensional matrix.

8. The three-dimensional matrix of claim 1 further comprising a means for enhancing the characteristics of the three-dimensional matrix.

9. The three-dimensional matrix of claim 1 wherein the fibroblasts produce a component selected from the group consisting of a collagen, a elastic fiber, a reticular fiber, a proteoglycan, and a hyaluronic acid.

10. The three-dimensional matrix of claim 1 wherein the fibroblasts are obtained from a mechanical disaggregation of a tissue, an enzymatic dissociation of a tissue, or fibroblast stem cells.

11. The three-dimensional matrix of claim 9 further comprising a means for stimulating the production of the collagen by the fibroblasts.

12. The three-dimensional matrix of claim 9 further comprising a means for inhibiting the rapid multiplication of the fibroblasts.

13. The three-dimensional matrix of claim 1 wherein the three-dimensional matrix is formed into a desired shape.

14. The three-dimensional matrix of claim 13 wherein the desired shape is selected from a group consisting of the shape of a blood vessel, a bone, a bone marrow, a brain, a cartilage, a connective tissue, an eye, an ear, an endocrine organ, an epithelial tissue, a muscle tissue, a hair, a heart, an intestine, a lung, a liver, a kidney, a mouth, a skin, a nail, a nose, a nerve tissue, a pancreas, a respiratory duct, a stomach, an urinary bladder, a spinal cord, a tongue, a tooth, and a reproductive organ.

15. The three-dimensional matrix of claim 13 wherein the desired shape is achieved through forming the three-dimensional matrix in a mold, wherein the mold has the desired shape.

16. The three-dimensional matrix of claim 13 wherein the desired shape is achieved through impregnating the three-dimensional matrix into a biocompatible scaffolding material, wherein the biocompatible scaffolding material forms the desired shape.

17. The three-dimensional matrix of claim 16 wherein the biocompatible scaffolding material is a biodegradable material or a non-biodegradable material.

18. The three-dimensional matrix of claim 1 wherein the three-dimensional matrix is used to culture cells or provide a scaffold for a living tissue equivalent.

19. A method of producing a three-dimensional matrix comprising mixing fibroblasts, thrombin, and fibrinogens or a blood plasma to form a mixture.

20. The method of claim 19 further comprising adding into the mixture a factor selected from the group consisting of a platelet, a factor XIII, a clotting factor, a fibronectin, a non-Willebrand factor, and a growth factor.

21. The method of claim 20 wherein the growth factor is selected the group consisting of TGF-β, PDGF, FGF, EGF, VEGF, IL-1, TNF and vitamin C.

22. The method of claim 19 wherein the fibroblasts produce a component selected from the group consisting of a collagen, an elastic fiber, a reticular fiber, a proteoglycan, and a hyaluronic acid.

23. The method of claim 19 wherein the fibroblasts are obtained from a mechanical disaggregation of a tissue, an enzymatic dissociation of a tissue, or fibroblast stem cells.

24. The method of claim 19 wherein the mixture is formed into a desired shape.

25. The method of claim 24 wherein the desired shape is selected from a group consisting of the shape of a blood vessel, a bone, a bone marrow, a brain, a cartilage, a connective tissue, an eye, an ear, an endocrine organ, an epithelial tissue, a muscle tissue, a hair, a heart, an intestine, a lung, a liver, a kidney, a mouth, a skin, a nail, a nose, a nerve tissue, a pancreas, a respiratory duct, a stomach, an urinary bladder, a spinal cord, a tongue, a tooth, and a reproductive organ.

26. The method of claim 24 wherein the desired shape of the mixture is achieved through forming the mixture in a mold, wherein the mold has the desired shape.

27. The method of claim 24 wherein the desired shape of the mixture is achieved through impregnating the mixture into a biocompatible scaffolding material, wherein the biocompatible scaffolding material forms the desired shape.

28. The method of claim 27 wherein the biocompatible scaffolding material is a biodegradable material or a non-biodegradable material.

29. The method of claim 19 wherein the three-dimensional matrix is used to culture cells or provide a scaffold for a living tissue equivalent.

30. A method of producing a three-dimensional matrix having a desired shape, comprising:
obtaining a mold having the desired shape, and
filing the mold with a mixture of fibroblasts, thrombin, and fibrinogens or a blood plasma.

31. The method of claim 30 wherein the desired shape is selected from a group consisting of the shape of a blood vessel, a bone, a bone marrow, a brain, a cartilage, a connective tissue, an eye, an ear, an endocrine organ, an epithelial tissue, a muscle tissue, a hair, a heart, an intestine, a lung, a liver, a kidney, a mouth, a skin, a nail, a nose, a nerve tissue, a pancreas, a respiratory duct, a stomach, an urinary bladder, a spinal cord, a tongue, a tooth, and a reproductive organ.

32. A method of producing a three-dimensional matrix having a desired shape, comprising:
mixing fibroblasts, thrombin, and fibrinogens or a blood plasma irito a mixture;
selecting a biocompatible material;
constructing the biocompatible materials to form the desired shape; and
impregnating the mixture into the biocompatible materials having the desired shape.

33. The method of claim 32 wherein the desired shape is selected from a group consisting of the shape of a blood vessel, a bone, a bone marrow, a brain, a cartilage, a connective tissue, an eye, an ear, an endocrine organ, an epithelial tissue, a muscle tissue, a hair, a heart, an intestine, a lung, a liver, a kidney, a mouth, a skin, a nail, a nose, a nerve tissue, a pancreas, a respiratory duct, a stomach, an urinary bladder, a spinal cord, a tongue, a tooth, and a reproductive organ.

34. The method of claim 32 wherein the biocompatible scaffolding material is a biodegradable material or a non-biodegradable material.

35. A living tissue equivalent comprising a fibrin matrix, fibroblasts, and cells, wherein the cells are not fibroblasts.

36. The living tissue equivalent of claim 35 wherein the fibrin matrix comprises fibrinogens and means for polymerizing and cross-linking the fibrinogens.

37. The living tissue equivalent of claim 35 wherein the fibrin matrix comprises fibrinogens and a thrombin.

38. The living tissue equivalent of claim 35 wherein the fibrin matrix comprises a blood plasma and a thrombin.

39. The living tissue equivalent of claim 35 further comprising a factor selected from the group consisting of a platelet, a factor XIII, a clotting factor, a fibronectin, a non-Willebrand factor, and a growth factor.

40. The living tissue equivalent of claim 39 wherein the growth factor is selected the group consisting of TGF-β, PDGF, FGF, EGF, VEGF, IL-1, TNF and vitamin C.

41. The living tissue equivalent of claim 35 further comprising a means for accelerating the formation of the living tissue equivalent.

42. The living tissue equivalent of claim 35 further comprising a means for enhancing the characteristics of the living tissue equivalent.

43. The living tissue equivalent of claim 35 wherein the fibroblasts produce a component selected from the group consisting of a collagen, a elastic fiber, a reticular fiber, a proteoglycan, and a hyaluronic acid.

44. The living tissue equivalent of claim 35 wherein the fibroblasts are obtained from a mechanical disaggregation of a tissue, an enzymatic dissociation of a tissue, or fibroblast stem cells.

45. The living tissue equivalent of claim 43 further comprising a means for stimulating the production of the collagen by the fibroblasts.

46. The living tissue equivalent of claim 43 further comprising a means for inhibiting the rapid multiplication of the fibroblasts.

47. The living tissue equivalent of claim 35 wherein the living tissue equivalent is formed into a desired shape.

48. The living tissue equivalent of claim 47 wherein the desired shape is selected from a group consisting of the shape of a blood vessel, a bone, a bone marrow, a brain, a cartilage, a connective tissue, an eye, an ear, an endocrine organ, an epithelial tissue, a muscle tissue, a hair, a heart, an intestine, a lung, a liver, a kidney, a mouth, a skin, a nail, a nose, a nerve tissue, a pancreas, a respiratory duct, a stomach, an urinary bladder, a spinal cord, a tongue, a tooth, and a reproductive organ.

49. The living tissue equivalent of claim 47 wherein the desired shape is achieved through forming the living tissue equivalent in a mold, wherein the mold has the desired shape.

50. The living tissue equivalent of claim 47 wherein the desired shape is achieve through impregnating the living tissue equivalent into a biocompatible scaffolding material, wherein the biocompatible scaffolding material forms the desired shape.

51. The living tissue equivalent of claim 50 wherein the biocompatible scaffolding material is a biodegradable material or a non-biodegradable material.

52. The living tissue equivalent of claim 35 wherein the cells are normal cells or diseased cells.

53. The living tissue equivalent of claim 52 wherein the diseased cell are cancer cells.

54. The living tissue equivalent of claim 35 wherein the cells are stem cells, wherein the stem cell are not fibroblast stem cells.

55. The living tissue equivalent of claim 54 wherein the stem cells are selected from the group consisting of epithelial stem cells, connective stem cells, muscle stem cells and nervous stem cells.

56. The living tissue equivalent of claim 35 wherein the cells are genetically modified through a transfection.

57. The living tissue equivalent of claim 56 wherein the transfection method is selected from the group consisting of a cationic lipid complex, a calcium phosphate coprecipitation, an electroporation, a DEAE-Dextran method, a viral infection, a microinjection, and a biolistic particle delivery.

58. The living tissue equivalent of claim 35 wherein the cells are selected from the group consisting of a blood vessel, a bone, a bone marrow, a brain, a cartilage, a connective tissue, an eye, an ear, an endocrine organ, an epithelial tissue, a muscle tissue, a hair, a heart, an intestine, a lung, a liver, a kidney, a mouth, a skin, a nail, a nose, a nerve tissue, a pancreas, a respiratory duct, a stomach, an urinary bladder, a spinal cord, a tongue, a tooth, and a reproductive organ.

59. A method of producing a living tissue equivalent comprising mixing thrombin, fibroblasts, cells, fibrinogens or a blood plasma, wherein the cells are not fibroblasts.

60. The method of claim 59 further comprising adding a factor selected from the group consisting of a platelet, a factor XIII, a clotting factor, a fibronectin, a non-Willebrand factor, and a growth factor.

61. The method of claim 60 wherein the growth factor is selected the group consisting of TGF-β, PDGF, FGF, EGF, VEGF, IL-1, TNF and vitamin C.

62. The method of claim 59 wherein the fibroblasts produce a component selected from the group consisting of a collagen, a elastic fiber, a reticular fiber, a proteoglycan, and a hyaluronic acid.

63. The method of claim 59 wherein the fibroblasts are obtained from a mechanical disaggregation of a tissue, an enzymatic dissociation of a tissue, or fibroblast stem cells.

64. The method of claim 59 wherein the mixture is formed into a desired shape.

65. The method of claim 64 wherein the desired shape is selected from a group consisting of the shape of a blood vessel, a bone, a bone marrow, a brain, a cartilage, a connective tissue, an eye, an ear, an endocrine organ, an epithelial tissue, a muscle tissue, a hair, a heart, an intestine, a lung, a liver, a kidney, a mouth, a skin, a nail, a nose, a nerve tissue, a pancreas, a respiratory duct, a stomach, an urinary bladder, a spinal cord, a tongue, a tooth, and a reproductive organ.

66. The method of claim 64 wherein the desired shape of the mixture is achieved through forming the three-dimensional matrix in a mold, wherein the mold has the desired shape.

67. The method of claim 64 wherein the desired shape of the mixture is achieve through impregnating the mixture a biocompatible scaffolding material, wherein the biocompatible scaffolding material forms the desired shape.

68. The method of claim 67 wherein the biocompatible scaffolding material is a biodegradable material or a non-biodegradable material.

69. The method of claim 59 wherein the cells are normal cells or diseased cells.

70. The method of claim 69 wherein the diseased cell are cancer cells.

71. The method of claim 59 wherein the cells are stem cells, wherein the stem cell are not fibroblast stem cells.

72. The method of claim 71 wherein the stem cells are selected from the group consisting of epithelial stem cells, connective stem cells, muscle tissue stem cells and nervous stem cells.

73. The method of claim 59 wherein the cells are genetically modified through a transfection.

74. The method of claim 73 wherein the transfection method is selected from the group consisting of a cationic lipid complex, a calcium phosphate coprecipitation, an electroporation, a DEAE-Dextran method, a viral infection, a microinjection, and a biolistic particle delivery.

75. The method of claim 59 wherein the cells are selected from the group consisting of a blood vessel, a bone, a bone marrow, a brain, a cartilage, a connective tissue, an eye, an ear, an endocrine organ, an epithelial tissue, a muscle tissue, a hair, a heart, an intestine, a lung, a liver, a kidney, a mouth, a skin, a nail, a nose, a nerve tissue, a pancreas, a respiratory duct, a stomach, an urinary bladder, a spinal cord, a tongue, a tooth, and a reproductive organ.

76. A method of producing a living tissue equivalent having a desired shape, comprising:
providing a mold having the desired shape; and
filing the mold with a mixture of cells, fibroblasts, thrombin, and fibrinogens or a blood plasma, wherein the cells are not fibroblasts.

77. The method of claim 76 wherein the desired shape is selected from a group consisting of the shape of a blood vessel, a bone, a bone marrow, a brain, a cartilage, a connective tissue, an eye, an ear, an endocrine organ, an epithelial tissue, a muscle tissue, a hair, a heart, an intestine, a lung, a liver, a kidney, a mouth, a skin, a nail, a nose, a nerve tissue, a pancreas, a respiratory duct, a stomach, an urinary bladder, a spinal cord, a tongue, a tooth, and a reproductive organ.

78. A method of producing a living tissue equivalent having a desired shape, comprising:
mixing cells, fibroblasts, thrombin, and fibrinogens or a blood plasma into a mixture, wherein the cells are not fibroblasts;
selecting a biocompatible material;
forming the biocompatible materials tinto the desired shape; and
impregnating the mixture into the biocompatible materials having the desired shape.

79. The method of claim 78 wherein the desired shape is selected from a group consisting of the shape of a blood vessel, a bone, a bone marrow, a brain, a cartilage, a connective tissue, an eye, an ear, an endocrine organ, an epithelial tissue, a muscle tissue, a hair, a heart, an intestine, a lung, a liver, a kidney, a mouth, a skin, a nail, a nose, a nerve tissue, a pancreas, a respiratory duct, a stomach, an urinary bladder, a spinal cord, a tongue, a tooth, and a reproductive organ.

80. The method of claim 78 wherein the biocompatible scaffolding material is a biodegradable material or a non-biodegradable material.

81. A method of testing the effect of an agent, comprising:
mixing a blood plasma or fibrinogen, thrombin, fibroblasts, and cells to form a living tissue equivalent, wherein the cells are not fibroblasts;
contacting the agent to the living tissue equivalent; and
evaluating the effect of the agent on the living tissue equivalent.

82. A method of testing the efficacy of a therapy treatment on a diseased tissue, comprising:
obtaining cells from the diseased tissue;
mixing a blood plasma or fibrinogens, thrombin, fibroblasts and the cells to form a living tissue equivalent, wherein the cells are not fibroblasts;
exposing the living tissue equivalent to the therapy treatment; and
evaluating the efficacy of the therapy treatment.

83. The method of claim 82 wherein the diseased tissue is a cancer tissue.

84. The method of claim 83 wherein the therapy treatment is a cancer therapy treatment.

85. A method of screening an agent, comprising:
mixing a blood plasma or fibrinogens, thrombin, fibroblasts, and cells to form a living tissue equivalent, wherein the cells are not fibroblasts; and
contacting the agent to the living tissue equivalent.

86. A method of expressing a desired protein in a tissue, comprising:
transfecting cells with genetic materials to become transfected cells, wherein the transfected cells are capable of expressing the desired protein;
mixing a blood plasma or fibrinogens, thrombin, fibroblasts, and the transfected cells to form a living tissue equivalent.

87. A method of producing a skin equivalent, comprising:
mixing a blood plasma or fibrinogen, thrombin, and fibroblasts to form a three-dimensional matrix;
growing the three-dimensional matrix in a growth media; and
plating skin cells on the surface of the three-dimensional matrix.

88. The method of claim 87 wherein the skin cells are keratinocytes.

89. The method of claim 87 wherein the skin cells are plated at a concentration of from 1×10² cells/cm³ to 1×10⁸ cells/cm³.

90. A method of producing a blood vessel equivalent, comprising:
mixing a blood plasma or fibrinogens, thrombin, first fibroblasts and smooth muscle tissue cells into a mixture;
casting the mixture around a cylindrical member;
plating second fibroblasts on the outer surface of the mixture; and
lining endothelial cells on the internal surface of the mixture.

91. A method of producing a bone tissue equivalent, comprising:
mixing a blood plasma or fibrinogens, thrombin, fibroblasts, and a demineralized bone powder.

92. A method of producing a bone equivalent, comprising:
mixing blood plasma, thrombin, fibroblasts, and undifferentiated cells, wherein the undifferentiated cells differentiate to osteoblasts.

93. A method of producing a cartilage tissue equivalent, comprising:
mixing blood plasma, thrombin, fibroblasts, and chondrocytes, or chondrocte stem cells.

94. A tissue microarray comprising a multiplicity of tissue equivalent blocks, wherein the tissue equivalent blocks are formed through slicing a living tissue equivalent comprising a mixture of a blood plasma or fibrinogens, thrombin, fibroblasts, and cells, wherein the cells are not fibroblasts.

95. A tissue microarray of claim 94 wherein the living tissue equivalent is embedded in a paraffin.

96. The tissue microarray of claim 94 wherein the living tissue equivalent is frozen.

97. The tissue microarray of claim 94 wherein the tissue equivalent blocks are used to probe a target selected from the group consisting of a DNA, a RNA, a protein, and a chemical agents.

98. The tissue microarray of claim 94 wherein the tissue equivalent blocks are used to identify in the living tissue equivalent blocks a molecule selected from the group consisting of a DNA, a RNA, and a protein.

99. A method of producing a tissue microarray, comprising:
mixing a blood plasma or fibrinogens, thrombin, fibroblasts, and cells into a living tissue equivalent, wherein the cells are not fibroblasts;
preparing the living tissue equivalent in a sliceable form;
slicing the living tissue equivalent into a multiplicity of tissue equivalent blocks; and
arraying the tissue equivalent blocks.

100. The method of claim 99 wherein the sliceable form is a paraffin embedding or a frozen section.

101. The method of claim 99 wherein the tissue equivalent blocks are arrayed manually or using a tissue microarraying instrument.

102. A method of replacing a tissue in a body, comprising:
mixing a blood plasma or fibrinogens, thrombin, fibroblasts, and cells into a living tissue equivalent, wherein the cells are not fibroblasts; and
implementing the living tissue equivalent in the body.
